(19) United States
(12) Patent Application Publication
(10) Pub. No.: US 2016/0106976 A1
(43) Pub. Date: Apr. 21, 2016

(54) DEVICE AND METHOD FOR TREATMENT OF SLEEP APNEA

(71) Applicant: Theodore R. Kucklick, Los Gatos, CA (US)

(72) Inventor: Theodore R. Kucklick, Los Gatos, CA (US)

(21) Appl. No.: 14/595,197

(22) Filed: Jan. 12, 2015

Related U.S. Application Data
(60) Provisional application No. 61/926,243, filed on Jan. 10, 2014.

(51) Int. Cl. A61N 1/36; A61N 1/05

(52) U.S. Cl. CPC A61N 1/3601; A61N 1/0548

(57) ABSTRACT

Method and device for the treatment of sleep apnea comprising a waterproof TENS (Transcutaneous electrical nerve stimulation) unit built into a mouth guard appliance. The device consists of a battery, a flex circuit, a micro controller and TENS generator, and electrode pad areas. The TENS unit evokes muscle contraction and trains muscles to improve muscle strength.

This application claims priority to U.S. Provisional Application 61/926,243 filed Jan. 10, 2014.

### FIELD OF THE INVENTION

The inventions described below relate to the field of treatment for sleep apnea.

### BACKGROUND OF THE INVENTIONS

One of the causes of obstructive sleep apnea and snoring is the loss of muscle tone in the airway. Breathing exercises have been prescribed to strengthen the muscles and treat sleep apnea, but these breathing exercises are strenuous and tedious and not all patients can and will do them.

### SUMMARY

Transcutaneous electrical nerve stimulation (TENS) is used to evoke muscle contraction and to train muscles and improve muscle strength. The device and methods described below provide for TENS type electro-stimulation to treat obstructive sleep apnea.

### BRIEF DESCRIPTION OF THE DRAWINGS

FIG. 1 shows a device for the treatment of sleep apnea.

FIG. 2 shows the device in use in a patient.

FIG. 3 shows a device for the treatment of sleep apnea.

FIG. 4 shows the device in use in a patient.

### DETAILED DESCRIPTION OF THE INVENTIONS

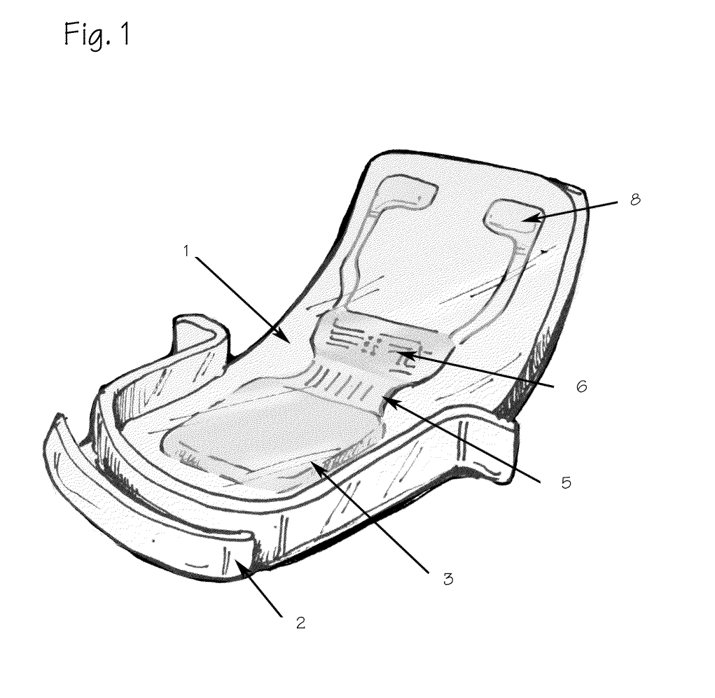

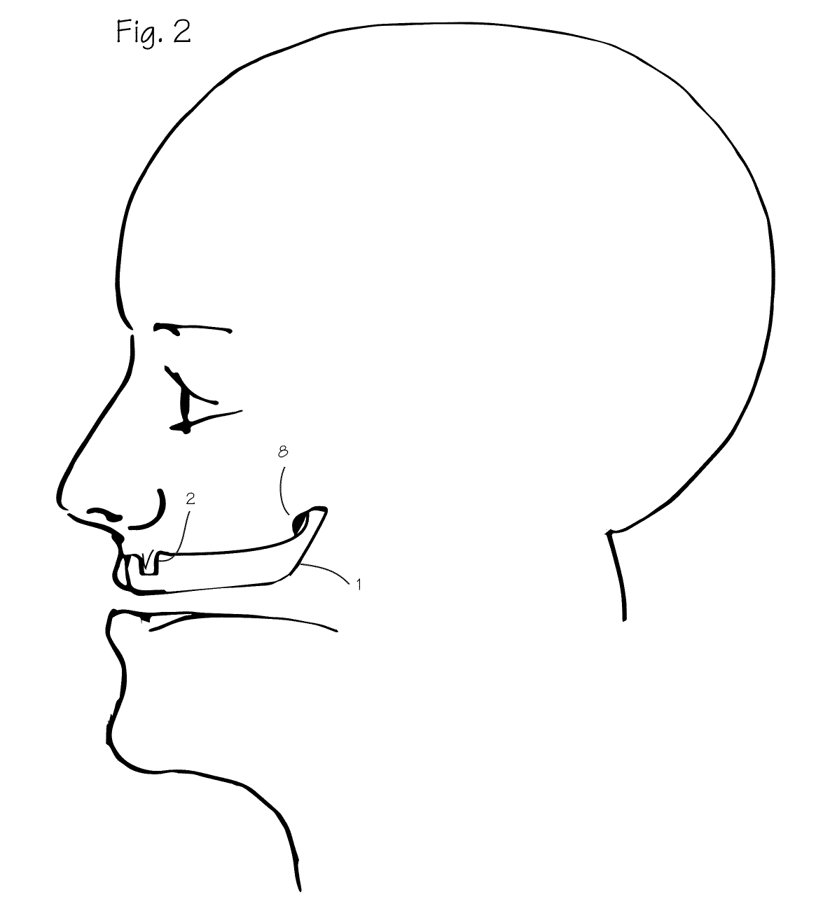

FIG. 1 shows a device for the treatment of sleep apnea. FIG. 2 shows the device in use on a patient. The device is a waterproof TENS (Transcutaneous electrical nerve stimulation) unit or similar electro-stimulation device that is built into a mouth guard appliance. It comprises the mouth guard 1 with a channel 2 configured for accepting the user's teeth, a battery 3, a palate extending from the channel 4, a flex circuit 5, a micro controller 6, electro-stimulation pulse generator 7, and electrode pad areas 8. The electronics are encased in a waterproof silicone enclosure 9. The mouth guard and palate are sized and dimensioned such that when the channel that accepts the teeth is disposed over the user's upper teeth, the electrodes are disposed over the target muscles or tissue overlying the target muscles. The mouth guard configuration is a convenient means for locating and holding the electrodes in place over the target muscles. Other means, similar to bridges or wire scaffolds, may be used, so long as they accomplish the goal of properly locating the electrodes.

The battery is sealed in the appliance and may be wirelessly rechargeable. The micro controller is programmable (preferably programmable through a wireless interface) so that a physician or the patient can program the device to the individual patient's tolerance for pulse power, frequency, and session length, or a prescribed power level and treatment regimen.

The electro-stimulation pulse generator 7 may be a Transcutaneous electrical nerve stimulation (TENS) device, but it may also be electrical muscle stimulation (EMS) device or electrotherapy generally, whether unipolar, bipolar, or using direct or alternating current. While TENS units transmit electrical signals to nerve endings, EMS units focus on the muscle. Both are effective for treatment of sleep apnea.

The current and waveform are of a type to produce the greatest exercising of the targeted muscle while minimizing irritation to the mucosal tissue surface, resulting in a treatment which is the least uncomfortable and thus the most tolerable for the patient. The frequency and power are such that they are enough to promote stimulation but are not at a level which will cause ablation of the tissue. TENS unit pain management parameters are pulse rate (1-250 Hz), pulse width (from 1-250 uS), and amplitude (0-100 mA). Neuromuscular Electrical Stimulation is used for muscle reeducation, spasticity reduction, strengthening, and other uses, with the range of effective frequencies are peak amperage (to the patient's tolerance level), pulse duration (50-300 microseconds), pulse frequency (1-200 pps) and pulse charge (≦10 mQ).

FIG. 2 shows the device placed in the anatomy. The device in inserted into the mouth, and the electrode pads contact the area of the oral cavity to be treated. The device may also be constructed with a plurality of electrodes to treat various muscles and muscle groups within the oral cavity. Target muscles include muscles of the airway, such as the styloglossus muscle, glossopharyngeus muscles, and the pharyngeal constrictor muscles; the group of muscles making up the soft palate including the tensor veli palatini, levator veli palatini, musculus uvulae, palatoglossus and the palatopharyngeus; and muscles underlying the oropharynx. The device may also be used to contract the muscles of the airway to open the airway to unobstructed breathing and to prevent snoring.

In use, the mouth guard is placed in the patient's mouth, such that the user's teeth are disposed within the teeth channel of the mouth guard and the electrodes are disposed in contact with tissue overlying the muscle or muscles of interest. The user then operates the pulse generator to generate muscle stimulating pulses which stimulate the target muscles. The user may adjust the power and frequency of the pulses to provide perceptible pulses, limited by the user's comfort level. The user may also operate the pulse generator for session lengths of several minutes, limited by the user's tolerance. The user may install and operate the device while awake, during waking hours, to train the target muscles, or install the device prior to sleep, and operate the device while sleeping at a low power level, optionally in conjunction with an apnea detection system (a microphone or flow sensor to detect lack of breathing or airway blockage) operable to detect airway blockage and initiate electro-stimulation pulses to cause contraction of airway muscles to open the airway by, for example, tensioning a collapsed soft palate.

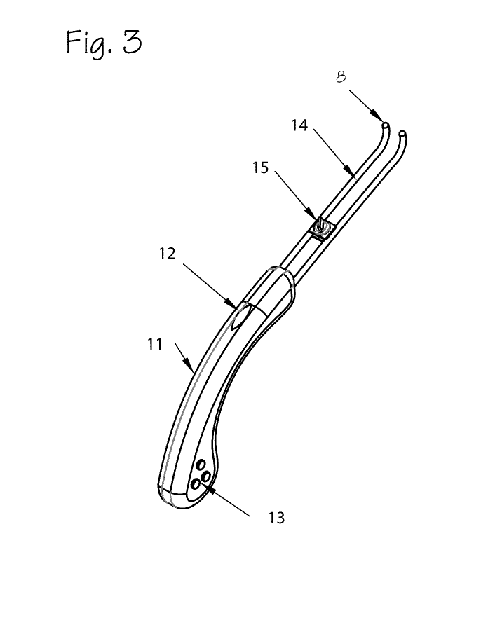

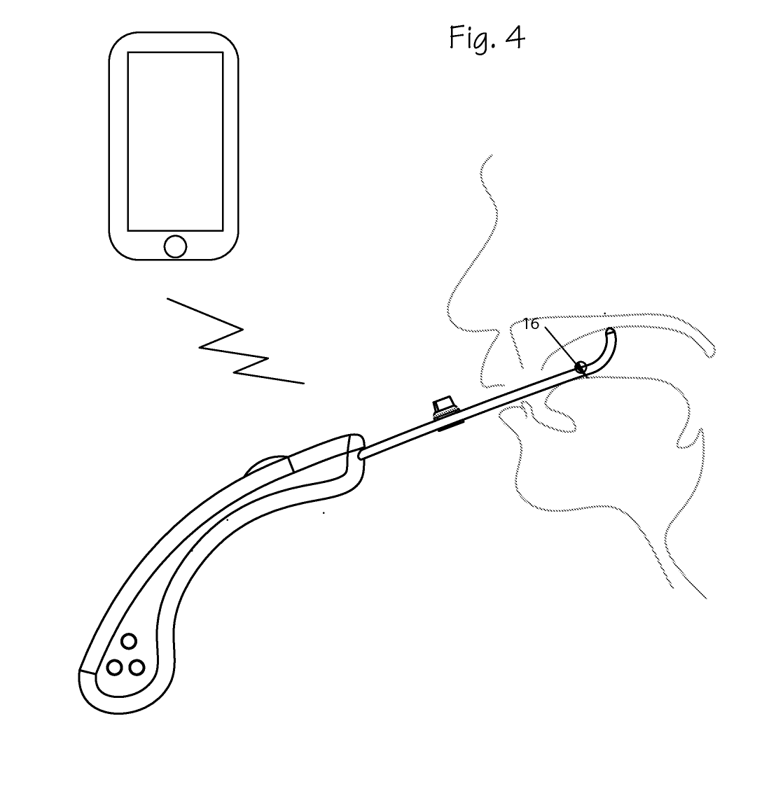

FIG. 3 shows a hand held device 10 for the treatment of sleep apnea. FIG. 4 shows the device in use on a patient. The hand held oral TENS/EMS device has an ergonomic handle 11 with a user controlled on/off switch 12 and powers and mode controls 13. Two tines 14 extend from the handle to deliver the TENS/EMS electrodes 8 to the targeted tissue. A spacing adjustment mechanism 15 allows for the length of the tines to be adjusted for delivery of the electrodes to the target tissue, based on the individual anatomy of the user. The device may be battery powered, rechargeable, or powered from a cord.

The device may have the ability to deliver moisturizing, lubricating, or conductive fluid to the patient's oral cavity to protect the patient's mucosal tissue or improve the efficiency of the electrodes. The device also monitors the impedance of the mucosal tissue and reduces or shuts off current if the tissue becomes too dry (high impedance).

The device is also capable of recording storing and transmitting data on usage to medical professional, by way of a dedicated wireless module or through a connection to a smartphone. The device may be programmable from its own user interface of a smartphone device. The device may have a wireless camera 16 that transmits to a video screen such as that on a smartphone to aid in the location of anatomical landmarks.

Thus, while the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other.

1. A device for the treatment of sleep apnea, said device comprising:
a housing adapted to be applied to the oral cavity of a patient;
an electrode assembly adapted for contact with the oral cavity of a patient;
said electrode assembly comprising an electrode; and
control circuitry operably connected to the electrode, said circuitry being selectively operable by the patient to generate electrical stimulation pulses and transmit those pulses through the electrodes to targeted muscle in the oral cavity of a patient.

2. The device of claim 1 wherein said targeted muscle of the oral cavity comprises the muscles of the soft palate, including but not limited to the tensor veli palatini, levator veli palatini, musculus uvulae, palatoglossus and the palatopharyngeus.

3. The device of claim 1 wherein said targeted muscle of the oral cavity comprises the muscles of the airway, including but not limited to the styloglossus muscle, glossopharyngeus muscles, and the pharyngeal constrictor muscles.

4. The device of claim 1 wherein said targeted muscle of the oral cavity includes muscles underlying the oropharynx.

5. The device of claim 1 wherein said housing comprises a mouth guard appliance carrying the electrodes.

6. The device of claim 1 wherein said housing comprises a hand held appliance carrying the electrode.

7. The device of claim 1 wherein said housing comprises a means for locating and holding the electrode in a place over the target muscles.

8. The device of claim 1 wherein said control circuitry further comprises delivering intermittent electrical stimulation pulses.

9. The device of claim 1 wherein said control circuitry further comprises delivering continuous electrical stimulation pulses.

10. A method for treating sleep apnea comprising the steps of:
mounting at least one electrode over the targeted muscle of the oral cavity;
generating a stimulation signal; and
delivering the stimulation signal to said at least one electrode to stimulate the targeted muscle of the oral cavity.

11. The method of claim 10 wherein said targeted muscle of the oral cavity comprises the muscles of the soft palate, including but not limited to the tensor veli palatini, levator veli palatini, musculus uvulae, palatoglossus and the palatopharyngeus.

12. The method of claim 10 wherein said targeted muscle of the oral cavity comprises the muscles of the airway, including but not limited to the styloglossus muscle, glossopharyngeus muscles, and the pharyngeal constrictor muscles.

13. The method of claim 10 wherein said targeted muscle of the oral cavity includes muscles underlying the oropharynx.

14. The method of claim 10 wherein said mounting step includes providing a mouth guard appliance carrying the electrodes, with the mouth guard appliance having a circuit means for generating the stimulation signal encased within the housing.

15. The method of claim 10 wherein said mounting step includes providing a hand held appliance carrying the electrodes, with the hand held appliance having a circuit means for generating the stimulation signal encased within the housing.

16. The method of claim 10 wherein said mounting step includes providing a means for locating and holding the electrode in a place over the target muscles, with the means having a circuit means for generating the stimulation signal encased within the housing.

17. The method of claim 10 wherein said delivering step comprises delivering an intermittent stimulation signal.

18. The method of claim 10 wherein said delivering step comprises delivering a continuous stimulation signal.
